(19) United States
(12) Patent Application Publication
(10) Pub. No.: US 2026/0174439 A1
(43) Pub. Date: Jun. 25, 2026

(54) INJECTING GLUCOSE SOLUTION TO PREVENT PROXIMAL MIGRATION OF INJECTED EMBOLIC SOLUTION DURING ENDOVASCULAR EMBOLIZATION TREATMENT IN A VESSEL

(71) Applicant: Neuravi Limited, Galway (IE)

(72) Inventors: David QUINN, Galway (IE); Karl KEATING, Galway (IE); Gillian GUNNING, Galway (IE); Ray McCARTHY, Galway (IE); Soraya SALINAS FERNANDEZ, Galway (IE); Michael SCANLON, Galway (IE)

(21) Appl. No.: 19/396,791

(22) Filed: Nov. 21, 2025

Related U.S. Application Data
(60) Provisional application No. 63/738,640, filed on Dec. 24, 2024.

(51) Int. Cl. A61B 17/12; A61B 17/00; A61M 25/00

(52) U.S. Cl. CPC A61B 17/12186; A61M 25/0029; A61B 2017/00778; A61B 2017/00951; A61B 2217/005; A61M 2025/0037; A61M 2025/0039; A61M 2025/0042; A61M 2202/0007; A61M 2202/049; A61M 2210/12

(57) ABSTRACT

Method and system for embolization treatment at a target site within a vessel using an endovascular embolization system. A microcatheter is navigated to the target site in the vessel. Embolic solution is injected into the vessel. Proximal migration in the vessel of the injected embolic solution is prevented by injecting into the vessel a glucose solution.

### CROSS REFERENCE TO RELATED APPLICATIONS

This application claims the benefit of priority under 35 U.S.C. § 119 to prior filed U.S. Provisional Ser. No. 63/738,640 , filed Dec. 24, 2024 (Attorney Docket No.: 243382.000590(NRV6149USPSP1)), the entire contents of which is hereby incorporated by reference in its entirety as if set forth in full herein.

### FIELD

The present disclosure generally relates to endovascular treatment to stop bleeding by injecting of a liquid embolic solution (e.g., glue) into the vasculature at the target site (e.g., source) of the bleeding. By way of example, the target site for the endovascular embolization treatment may be the middle meningeal artery (MMA). In particular, the present disclosure is directed to an improved endovascular embolization treatment that injects glucose solution into the vessel to prevent undesirable proximal migration resulting from back pressure build-up of the injected embolic solution.

### BACKGROUND

Endovascular treatment is widely used to stop bleeding (e.g., embolization) at a target site in a vessel. Embolization treatment may occur anywhere in the body, for example, in the middle meningeal artery (MMA). During endovascular embolization treatment an embolic solution (e.g., solution of an embolic agent (e.g., n-butyl-cyanoacrylate (n-BCA)) and an oil) may be injected into the vessel at the target site (e.g., bleeding site in the wall of the vessel). As a result of back pressure build-up, the injected embolic solution undesirably migrates in a proximal direction resulting in one or more problems: (i) potential risk of unintentional occlusion of vessels at a location proximally of the target site; (ii) clogging of the lumen of the microcatheter with the injected embolic solution preventing tracking over a guidewire received therein; (iii) and/or adherence of the injected embolic solution to the exterior surface (i.e., outer wall) of the microcatheter.

It is therefore desirable to develop an improved endovascular embolization treatment that prevents or minimizes risk of proximal migration of the injected embolic solution while also preventing adherence of the embolic solution in the lumen of the microcatheter and to the exterior surface of the microcatheter.

### SUMMARY

An aspect of the present disclosure relates to an improved endovascular embolization treatment preventing or minimizing risk of proximal migration of the injected embolic solution.

Another aspect of the present disclosure is directed to an improved endovascular embolization treatment preventing adherence of the injected embolic solution in the lumen of the microcatheter when tracking over the guidewire.

While still another aspect of the present disclosure relates to an improved endovascular embolization treatment preventing adherence to the exterior surface of the microcatheter of the injected embolic solution.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1A is side view of an example endovascular embolization system with a dual lumen microcatheter in accordance with the present disclosure navigated to a target site in a vessel; wherein in the longitudinal/axial direction the outlet port of the glucose solution lumen is disposed proximally relative to the outlet port of the embolic solution lumen;

FIG. 1B is a longitudinal cross-sectional view of an example eccentrically arranged dual lumen microcatheter in the endovascular embolization system in FIG. 1A;

FIG. 1C is a radial cross-sectional view along lines 1C-1C of FIG. 1B;

FIG. 1D is a longitudinal cross-sectional view of an alternative example concentrically arranged dual lumen microcatheter in the endovascular embolization system in FIG. 1A;

FIG. 1E is a radial cross-sectional view along lines 1E-1E of FIG. 1D;

FIG. 2A is side view of another example endovascular embolization system with an eccentrically arranged dual lumen microcatheter in accordance with the present disclosure navigated to a target site in a vessel; wherein in the longitudinal/axial direction the outlet ports of the respective embolic solution lumen and glucose solution lumen are substantially aligned with one another coinciding with the distal end/tip of the microcatheter;

FIG. 2B is a longitudinal cross-sectional view of the eccentric arrangement of the dual lumen microcatheter in the endovascular embolization system in FIG. 2A;

FIG. 2C is a radial cross-sectional view along lines 2C-2C of FIG. 2B;

FIG. 3A is side view of still another example endovascular embolization system with a concentrically arranged dual lumen microcatheter in accordance with the present disclosure navigated to a target site in a vessel; wherein in the longitudinal direction the outlet ports of the respective embolic solution lumen and glucose solution lumen are substantially aligned with one another coinciding with the distal end/tip of the microcatheter; and within the vessel the injected glucose solution radially outward (i.e., abluminal) relative to the centrally injected embolic solution;

FIG. 3B is a longitudinal cross-sectional view of the concentric arrangement of the dual lumen microcatheter in the endovascular embolization system in FIG. 3A;

FIG. 3C is a radial cross-sectional view along lines 3C-3C of FIG. 3B;

FIG. 4A is side view of still another example endovascular embolization system with a concentrically arranged dual lumen microcatheter in accordance with the present disclosure navigated to a target site in a vessel; wherein in the longitudinal direction the outlet ports of the respective embolic solution lumen and glucose solution lumen are substantially aligned with one another coinciding with the distal end/tip of the microcatheter; and within the vessel the injected embolic solution is disposed radially outward (i.e., abluminal) relative to the centrally injected glucose solution;

FIG. 4B is a longitudinal cross-sectional view of the concentric arrangement of the dual lumen microcatheter in the endovascular embolization system in FIG. 4A;

FIG. 4C is a radial cross-sectional view along lines 4C-4C of FIG. 4B;

FIG. 5A is a side view of still another example endovascular embolization system with an eccentrically arranged dual lumen microcatheter in accordance with the present disclosure navigated to a target site in a vessel; wherein in the longitudinal direction the outlet port of the glucose solution lumen is disposed proximally relative to the outlet port of the embolic solution lumen;

FIG. 5B is a longitudinal cross-sectional view of the eccentric arrangement of the dual lumen microcatheter in the endovascular embolization system in FIG. 5A;

FIG. 5C is a radial cross-sectional view along lines 5C-5C of FIG. 5B;

FIG. 6A is a side view of while yet another example endovascular embolization system with a triple lumen microcatheter in accordance with the present disclosure navigated to a target site in a vessel; wherein the three lumens (e.g., an embolic solution lumen, a glucose solution lumen and an aspiration lumen) are arranged side-by-side extending parallel to one another in the longitudinal/axial direction;

FIG. 6B is a radial cross-sectional view along lines 6B-6B of FIG. 6A;

FIG. 7A is side view of still another example endovascular embolization system with two telescopically arranged microcatheters in accordance with the present disclosure navigated to a target site in a vessel; wherein in the longitudinal direction the distal end of the inner microcatheter delivering the glucose solution extends distally in the longitudinal/axial direction relative to the distal end of the outer microcatheter delivering the embolic solution; within the vessel the injected embolic solution is disposed radially outward (i.e., abluminal) relative to the centrally injected glucose solution;

FIG. 7B is a longitudinal cross-sectional view of the concentric arrangement of the inner catheter arranged telescopically within the outer microcatheter of the endovascular embolization system in FIG. 7A;

FIG. 7C is a radial cross-sectional view along lines 7C-7C of FIG. 7B;

FIG. 7D is side view of yet another example endovascular embolization system with two telescopically arranged microcatheters in accordance with the present disclosure navigated to a target site in a vessel; wherein in the longitudinal/axial direction the distal end of the inner microcatheter delivering the embolic solution terminates proximally relative to the distal end of the outer microcatheter delivering the glucose solution; within the vessel the injected glucose solution is disposed radially outward (i.e., abluminal) relative to the centrally injected embolic solution;

FIG. 7E is a longitudinal cross-sectional view of the telescopic arrangement of the two microcatheters in the endovascular embolization system in FIG. 7D;

FIG. 7F is a radial cross-sectional view along lines 7F-7F of FIG. 7E;

FIG. 8A is side view of an example endovascular embolization system with two telescopically arranged microcatheters in accordance with the present disclosure navigated to a target site in a vessel; wherein in the longitudinal/axial direction the distal end of the inner microcatheter delivering the embolic solution extends distally beyond the distal end of the outer microcatheter delivering the glucose solution; within the vessel the injected glucose solution is disposed radially outward (i.e., abluminal) relative to the centrally injected embolic solution;

FIG. 8B is a side view of an example telescopic dispensing system for advancing the inner microcatheter while the outer microcatheter remains stationary of the example endovascular embolization system of FIG. 8A; wherein the microcatheters are depicted in a longitudinal/axial cross-sectional view illustrating their concentric arrangement;

FIG. 8C is a radial cross-sectional view along lines 8C-8C of FIG. 8B;

FIG. 9A is a longitudinal cross-sectional view of an example dual channel dispensing system including a single barrel syringe having dual concentric channels feeding into a single luer attachment or hub attached to a proximal end of a microcatheter (e.g., dual lumen or single lumen) for in tandem injection of the embolic solution and glucose solution;

FIG. 9B is a longitudinal cross-sectional view of another exemplary dual channel dispensing system including a dual barrel syringe feeding into respective channels of a single luer attachment or hub that, in turn, is attached to a proximal end of a single lumen microcatheter for administering embolic solution and glucose solution;

FIG. 9C is a distal end view of the single lumen microcatheter in the dual channel dispensing system of FIG. 9B;

FIG. 9D is a longitudinal cross-sectional view of another exemplary dual channel dispensing system including a dual barrel syringe feeding into respective channels of a single luer attachment or hub that, in turn, is attached to a proximal end of a single lumen microcatheter for administering embolic solution and glucose solution, wherein the distal opening of the channel in the hub transporting glucose solution coincides in the longitudinal direction with the distal opening of the channel in the hub delivering embolic solution;

FIG. 9E is a longitudinal cross-sectional view of yet another exemplary dual channel dispensing system including a dual barrel syringe feeding into respective channels of a single luer attachment or hub that, in turn, is attached to a proximal end of a dual concentric lumen microcatheter for administering embolic solution and glucose solution, wherein the distal opening of the channel in the hub transporting glucose solution coincides in the longitudinal direction with the distal opening of the channel in the hub delivering embolic solution;

FIG. 10 is a side view of an example dual lumen hub with dual lure connectors fitted with respective ampules of preloaded, premixed of embolic solution and glucose solution, respectively, injectable in tandem into the microcatheter (e.g., dual lumen or single lumen);

FIG. 11A is a side view of an example dual channel hub for manually controllable sequenced dispensing from multiple supply canisters of respective embolic solution and glucose solution into the microcatheter;

FIG. 11B is a side view of an example dual channel hub for electronically programable sequenced dispensing from multiple supply canisters of respective embolic solution and glucose solution into the microcatheter; and

FIG. 12 is an example hub connectable to the proximal end of the microcatheter (single lumen or dual lumen); wherein the hub is fitted with lure connectors fitted with corresponding ampules sealed via a removable locking tab, the sealed ampules containing preloaded, premixed embolic solution (e.g., embolic agent and oil) and preloaded, premixed glucose solution (e.g., dextrose approximately 5% in deionized water), respectively; and

FIG. 13 is a flow chart of the method of operation of the endovascular embolization system in accordance with the present disclosure wherein glucose solution is injected into the vessel to prevent proximal migration of the injected embolic solution.

### DETAILED DESCRIPTION

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 99%.

As used herein, the term “microcatheter” is a catheter having a diameter that is small in comparison to catheters in cardiovascular applications, i.e. 8 French or less.

As used herein, the terms “tubular” and “tube” are to be construed broadly and are not limited to a structure that is a right cylinder or strictly circumferential in cross-section or of a uniform cross-section throughout its length. For example, a tubular structure or system is generally illustrated as a substantially right cylindrical structure. However, the tubular system may have a tapered or curved outer surface without departing from the scope of the present disclosure.

Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

Various example embolization treatment systems described and illustrated herein in accordance with the present disclosure stop bleeding at a desired target site (e.g., in the Middle Meningeal Artery (MMA)) by injecting into the vessel an embolic solution (e.g., embolic agent such as n-butyl-cyanoacrylate (n-BCA) and oil in any desired ratio). Despite the advantages associated with such endovascular treatment, one significant drawback is back pressure from build-up of the injected embolic solution resulting in undesirable migration of the injected embolic solution within the vessel in a proximal direction relative to the target site in which it was administered via the outlet port of the microcatheter. Several concerns arise from the proximal migration in the vessel of the injected embolic solution. Vessel(s) downstream in the vasculature relative to the target site of the injected embolic solution may unintentionally and undesirably become occluded from proximal migration of the injected embolic solution. In addition, the injected embolic solution that migrates proximally adheres to the exterior surface of the microcatheter hampering or preventing withdraw from the body. Still further the injected embolic solution may clog the lumen of the microcatheter prohibiting tracking of the guidewire therein. The issue of proximal migration of the injected embolic solution in the vessel is addressed by the present endovascular embolization system and method of treatment by the simultaneous (i.e., in tandem or at the same time) or non-simultaneous (i.e., sequential, independent or not at the same time) injection, dispensing or administering of a glucose solution (e.g., dextrose, preferably approximately 5%, in deionized water) into the vessel. It is noted that the glucose solution cannot include anything (e.g., saline) that would interact with and cause to cure the embolic solution. In addition to overcoming the issue of proximal migration of the injected embolic solution, in certain configurations or examples illustrated herein and described below, the injection of glucose solution imparts a force pushing the injected embolic solution further distally into the vessel beyond the limited reach of the microcatheter.

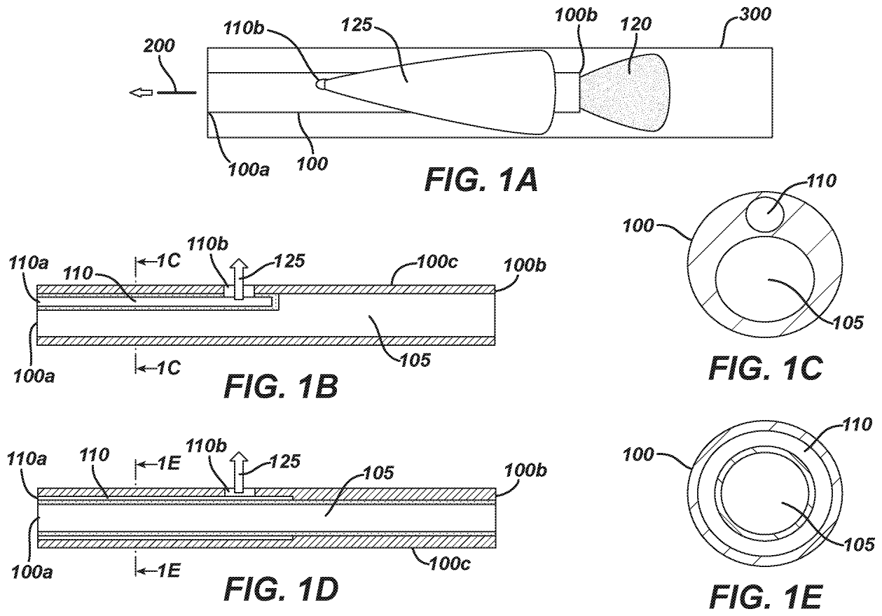

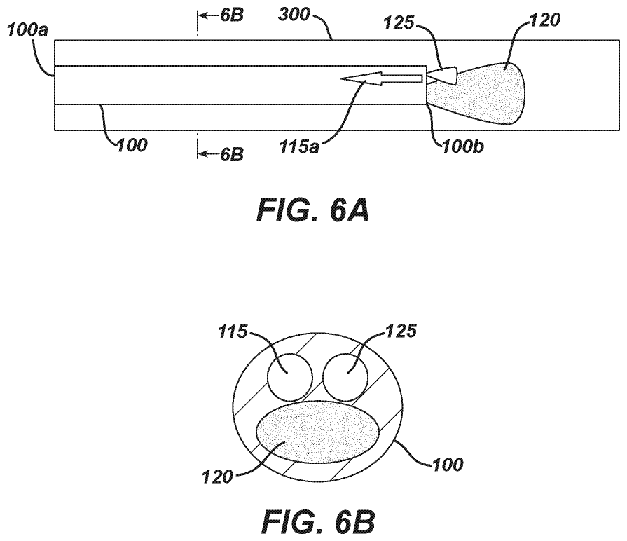

Several illustrative example configurations of an endovascular embolization system delivering the injected embolic solution into the vessel using a multi-lumen microcatheter 100 are disclosed herein (e.g., a dual lumen microcatheter 100 having two lumen 105, 110 separate and independent of one another). For example, microcatheter 100 depicted in FIGS. 1A-1E includes an embolic solution lumen 105 that tracks over a guidewire 200 receivable therein during navigation of the microcatheter 100 to the target site in the vessel and subsequent delivery of the embolic solution 120. Glucose solution 125 is delivered through a glucose solution lumen 110 separate from the embolic solution lumen 105. More than two lumens are contemplated, for example, an optional third lumen (e.g., dedicated guidewire lumen and/or aspiration lumen) separate and independent from each the embolic solution lumen 105 and the glucose solution lumen 110. For instance, the microcatheter illustrated in FIGS. 6A & 6B includes an aspiration lumen 115, as described in detail further below. As is evident from the side view of the first example dual lumen microcatheter 100 in FIG. 1A, the embolic solution lumen 105 extends in the longitudinal/axial direction from the inlet port at the proximal end 100a of the microcatheter to the outlet port at the distal end 100b of the microcatheter defining an outer wall 100c of the microcatheter extending therebetween. Arranged eccentrically of the embolic solution lumen 105, the glucose solution lumen 110 has an inlet port 110a and a side outlet port 110b defined in the outer wall 100c of the microcatheter 100. The side outlet port 110b of the glucose solution lumen 110 is arranged in the longitudinal/axial direction proximally of the outlet port of the embolic solution lumen 105 (coinciding or aligned with the distal end/tip of the microcatheter 100). As is evident from the side view of FIG. 1A the embolic solution 120 is injected via the outlet port of the embolic solution lumen 105 (coinciding or aligned with the distal end/tip 100b of the microcatheter 100) at the target site of the bleeding the vessel to be treated. Proximally thereof the glucose solution 125 dispensed from the side outlet port 110b of the glucose solution lumen 110 creates a projection of fluid region (e.g., a cone or funnel) in the vessel predominantly in a proximal direction (albeit some may flow in a proximal direction) thereby preventing proximal migration of the injected embolic solution 120. Different arrangements of the respective embolic solution lumen 105 and glucose lumen 110 are possible for the microcatheter in FIG. 1A. An eccentric arrangement of the embolic solution lumen 105 and glucose solution lumen 110 is shown in the example longitudinal/axial and radial cross-sectional views of FIGS. 1B & 1C, respectively. Alternatively, a concentric arrangement of the embolic solution lumen 105 and glucose solution lumen 110 is depicted in the example longitudinal/axial and radial cross-sectional views of FIGS. 1D & 1E, respectively. The size, shape and arrangement of each of the lumens 105, 110 may be selected, as desired. Injection of the embolic solution 120 and the glucose solution 125 may be either simultaneously (i.e., in tandem or at the same time) or non-simultaneously (i.e., sequentially, independently or not at the same time). In the case of non-simultaneous injection, the order, timing, duration and volume dispensed of each of these two solutions 120, 125 may be selected, as desired. The glucose solution may be injected at different ranges and/or may include a radiopaque element to visualize the presence of the injected glucose solution before administering the embolic solution. If too much glucose solution is injected, any excess may be advanced/pushed in a distal direction by the injected embolic solution. In one example, the physician may first inject a quantity of embolic solution followed by injection of glucose solution to push the injected embolic solution further in a distal direction prior to curing. In an alternative example, the physician may first administer a relatively small amount of glucose solution without extending too far distal of the catheter tip, and subsequently administering as a second injection the embolic solution following up thereafter with a third injection of glucose solution to advance/push the injected embolic solution still further in a distal direction. In FIGS. 1A-1E, depicting a moment in time, the glucose solution 125 minimizes the extent to which or prevents altogether the embolic solution 120 from adhering to an exterior surface of the microcatheter. Another advantage of the example in FIGS. 1A-1E is the injected glucose solution 125 imparts a force on pushing the injected embolic solution 120 distally further into the vessel/vasculature beyond those regions or areas too narrow to accommodate or able to be reached by the microcatheter 100. In addition, the injected embolic solution 120 is prevented from migrating proximally in the vessel due to back pressure build-up counterbalanced by the injected glucose solution 125.

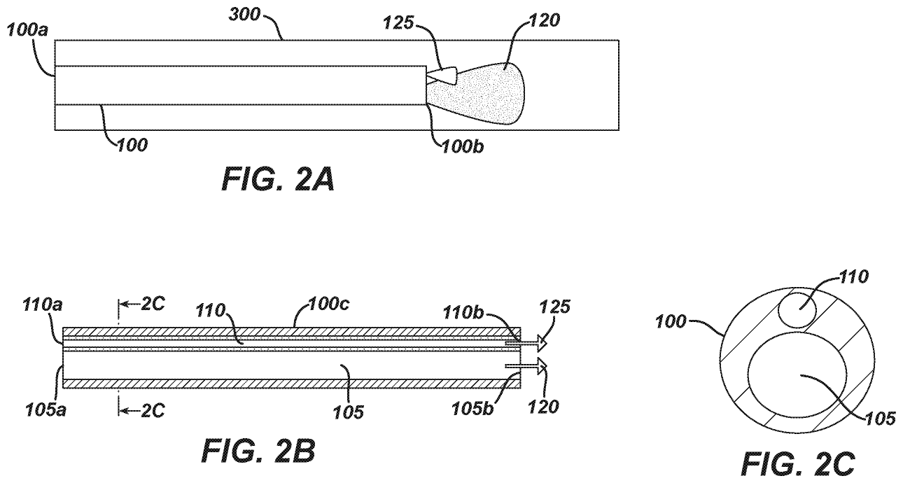

In a next example in FIGS. 2A-2C, the embolic and glucose lumens 105, 110, respectively, are arranged eccentrically with their respective outlet ports 105b, 110b coinciding or aligned with one another in the longitudinal/axial direction at the distal end/tip 100b of the microcatheter 100. Thus, the injected embolic and glucose solutions, 120, 125, respectively, form side-by-side regions of projected fluid (e.g., cone shape regions) proximate one another in the vessel 300 (FIG. 2A). The injected glucose solution 125 imparts a force on pushing the injected embolic solution 120 further distally into the vessel 300 beyond the reach where the microcatheter 100 may be accommodated. Where present, the injected glucose solution 125 prevents the injected embolic solution 120 from adhering to the exterior surface of the outer wall 100c of the microcatheter 100. Specifically, it takes time for the injected embolic solution to cure (e.g., several minutes) so the physician may first inject the embolic solution and then advance/push the uncured injected embolic solution distally by subsequent administration of the glucose solution. Proximal migration resulting from back pressure build up of the injected embolic solution is minimized or prevented as a result of the injected glucose solution 125. It is noted that the region of injected glucose solution need not completely fill or occlude the vessel if a sufficient amount of glucose solution is administered. By controlling flow rates, for example, glucose solution may be administered at a first flow rate to fill around the catheter tip while simultaneously administering the embolic solution at a second flow rate higher than the first flow rate to advance/push the embolic solution through the flow of glucose solution.

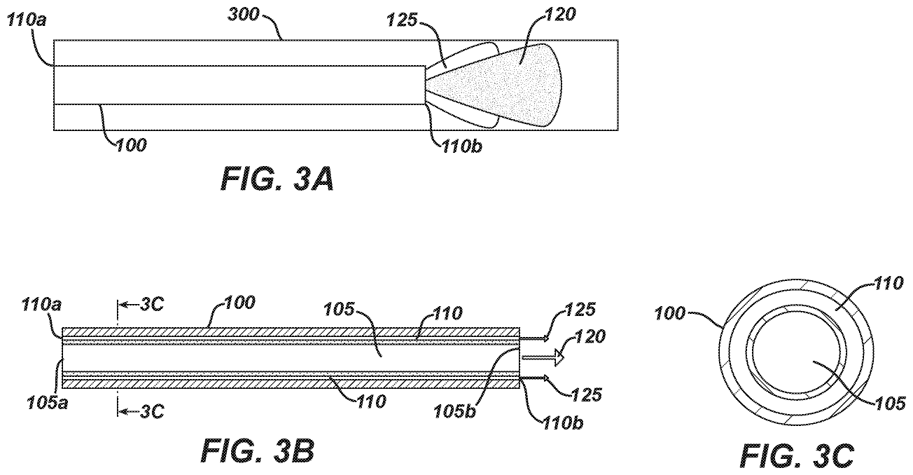

The dual lumens in the example microcatheter of FIGS. 3A-3C are arranged concentrically of one another (as shown in the longitudinal/axial and radial cross-sectional view of FIGS. 3B & 3C, respectively). Specifically, the embolic solution (i.e., glue) 120 is delivered via the concentric inner lumen 105 (e.g., embolic solution lumen) while the glucose solution 125 is delivered through the concentric outer lumen 110 (e.g., glucose solution lumen). Distal outlet ports 105b, 110b, of the respective lumens 105, 110 coincide and align with one another at the distal end/tip 100b of the microcatheter 100. Injection of the embolic and glucose solutions 120, 125, respectively, preferably occur in tandem (i.e., simultaneously or at the same time). With this concentric arrangement of the lumens 105, 110, the injected embolic solution 120 forms a central (inner) projection of fluid region (e.g., inner cone region) in the vessel while the injected glucose solution 125 forms an abluminal outer projection of fluid region (e.g., funnel region) 360 degrees surrounding that of the central (inner) projection of fluid region (e.g., cone) of injected embolic solution 120. Injected glucose solution 125 in the vessel 300 prevents proximal migration of the injected embolic solution 120 by counterbalancing the back pressure build-up of the injected embolic solution 120. Moreover, the injected central (inner) projection of fluid region (e.g., cone region) of embolic solution 120 is prevented from adhering to the exterior surface of the outer wall 100c of the microcatheter 100 via the abluminal outer funnel of injected glucose solution 125. Also, the injected glucose solution 125 aids in pushing the injected embolic solution 120 further distally into the vessel beyond the reach of the microcatheter 100.

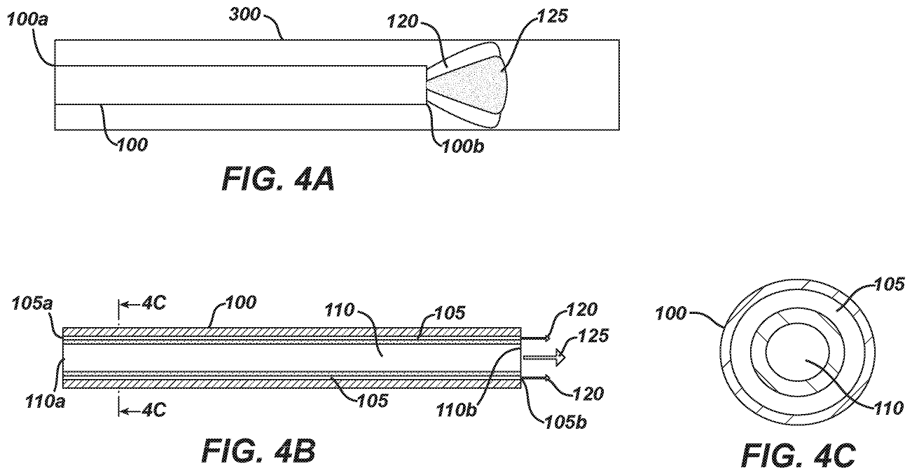

Like that in FIG. 3A-3C, the embolic solution lumen 105 and the glucose solution 110 in the dual lumen microcatheter 100 of FIG. 4A-4C are also arranged concentrically of one another. However, in FIG. 3A-3C the outer concentric glucose solution lumen 110 is arranged radially outward of the inner concentric embolic solution lumen 105, whereas the microcatheter in FIGS. 4A-4C presents the reverse arrangement with the outer concentric embolic solution lumen 105 arranged radially outward of the inner concentric glucose solution lumen 110. Referring to the side profile of the microcatheter 100 at a target site in the vessel 300 represented in FIG. 4A, injection in tandem of the solutions 120, 125 in the vessel 300 forms an abluminal outer projection region (e.g., funnel) of injected embolic solution 120 disposed radially outward of a central (inner) projection of fluid region (e.g., cone) of the glucose solution 125. If injection of glucose solution is continued after stopping injection of embolic solution, then the glucose solution would flush the embolic solution away from the exterior surface of the catheter. This central (inner) projection of fluid region (e.g., cone) of injected glucose solution 125 advantageously prohibits the abluminal (radially outward) outer projection region (e.g., funnel) of injected embolic solution 120 from otherwise entering into the outlet port 110b and clogging the glucose solution lumen 110 freely tracking over the guidewire 200. Furthermore, such wedge-shape outer projection region (e.g., funnel) of the injected embolic solution 120 in direct physical contract with the vessel 300 prevents back flush of the injected glucose solution 125.

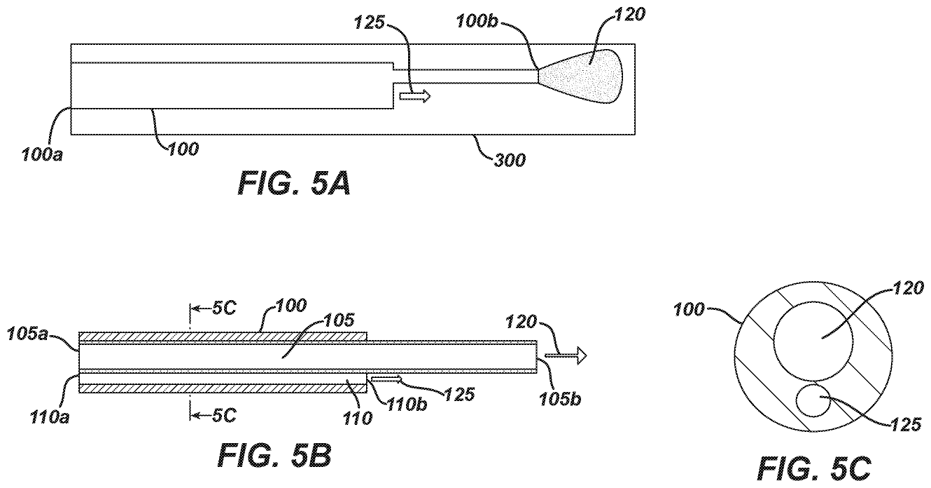

Another example dual lumen microcatheter is shown in FIGS. 5A-5C with the embolic solution lumen and glucose solution lumen 105, 110, respectively, arranged eccentrically of one another. The outlet port 110b of the glucose solution lumen 110 is disposed in the longitudinal/axial direction proximally of the outlet port 105b of the embolic solution lumen 105 similar to that of FIGS. 1A-1E. However, in FIGS. 1A-1E the side outlet port 110b of the glucose solution lumen 110 is defined along the outer surface 100c of the microcatheter 100, whereas in FIGS. 5A-5C the outlet port 110b exits from the distal end of the glucose solution lumen 110. In this modified configuration in FIG. 5A-5C, both outlet ports 105b, 110b of the respective lumens 105, 110 are parallel to one another in the longitudinal/axial direction.

As previously mentioned, the microcatheter in accordance with the endovascular embolization system of the present disclosure may have more than two lumen separate and independent of one another. Aspiration may be applied via a dedicated aspiration lumen 115 separate from the embolic and glucose lumens 105, 110, respectively. Fluid 115a (represented by the arrow in FIG. 6A) aspirated into the aspiration lumen 115 may include blood, excess injected embolic solution 120 and/or excess injected glucose solution 125. Accordingly, aspiration of fluid (including excess injected embolic solution 120) is yet another technique in accordance with the present disclosure for counterbalancing back pressure build-up of and hence preventing proximal migration of the injected embolic solution 120 and unintentional occlusion of vessels located proximally of the target site. Furthermore, aspiration of fluid 115a from the vessel 300 prevents the injected embolic solution 120 from adhering to the exterior surface of the microcatheter 100. FIGS. 6A & 6B depict an example triple lumen microcatheter including an embolic solution lumen 105, a glucose solution lumen 110 and an aspiration lumen 115, arranged side-by-side parallel to each other in the longitudinal/axial direction. Embolic solution lumen 105 in the example microcathet1er of FIGS. 6A & 6B represents a main lumen of the microcatheter, however, the size, shape and/or arrangement of each lumen 105, 110, 115 may be modified, as desired. All three operations (e.g., injection of embolic solution 120, injection of glucose solution 125 and aspiration of fluid 115a from the vessel) may occur simultaneously (i.e., in tandem or at the same time) or non-simultaneously (e.g., independently, sequentially or not at the same time). Therefore, all three operations may occur in tandem (i.e., simultaneously). Alternatively, any two of the three operations may take place simultaneously (e.g., simultaneous injection of embolic solution 120 and glucose solution 125), while aspiration occurs non-simultaneously or independently. Still further it is possible for all three operations to occur sequentially or independently of one another, in any desired order. The aspiration and injected glucose solution 125 prevents the injected embolic solution 120 from adhering to the exterior surface of the outer wall 100c of the microcatheter 100. Also, the injected glucose solution 125 pushes the injected embolic solution 120 further distally in the vessel 300 beyond the reach of the microcatheter 100. This exemplary configuration of the microcatheter in FIGS. 6A & 6B prevents proximal migration of the injected embolic solution 120 both by aspiration of fluid 115a from the vessel 300 in combination with the injection of the glucose solution 125 counterbalancing back pressure buildup of the injection of the embolic solution 120.

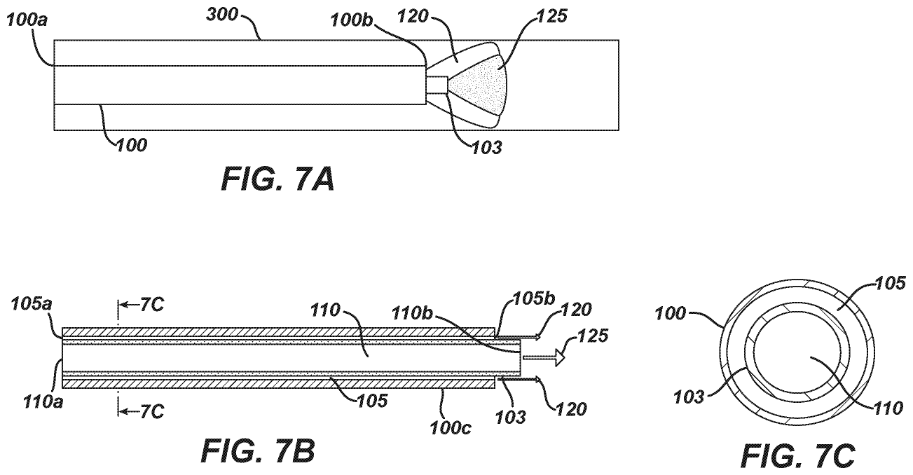

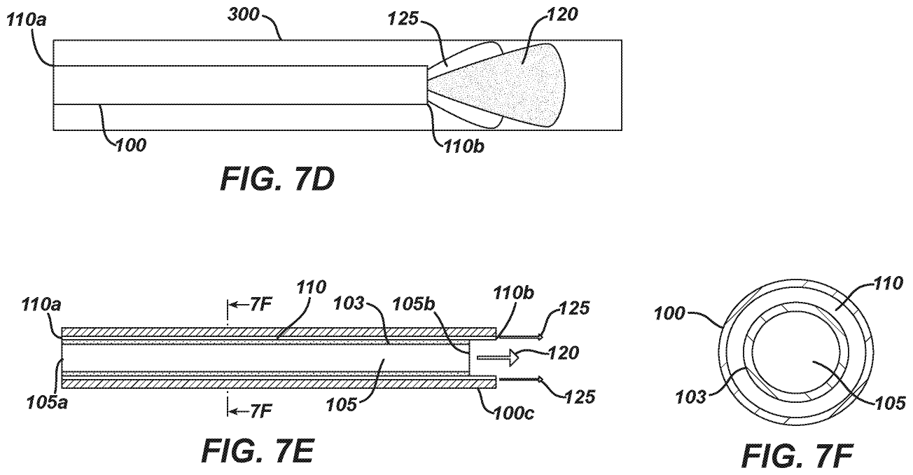

FIGS. 7A-7F present yet another example endovascular embolization system employing two telescopically arranged microcatheters (e.g., an inner microcatheter 103 and an outer microcatheter 100 disposed radially outward relative to the inner microcatheter 103). In one example depicted in FIGS. 7A-7C, the inner microcatheter 103 has defined therein a single lumen 110 for delivery of the glucose solution 125, while the embolic solution 120 is delivered via a channel defined between the exterior surface/outer wall of the inner microcatheter 103 and the inner wall of the outer microcatheter 100. Arrangement of the two microcatheters 100, 103 preferably is with the distal end/tip 103b of the inner microcatheter 103 disposed in the longitudinal/axial direction distally relatively to the distal end/tip 105b of the outer microcatheter 100. Referring to FIG. 7A, within the vessel 300 the injected embolic solution 120 forms an abluminal outer projection of fluid region (e.g., wedge-shape funnel) with the injected glucose solution 125 forming a central (inner) projection of fluid region (e.g., cone) disposed radially inward relative thereto. The embolic solution 120 and glucose solution 125 are preferably injected simultaneously (i.e., in tandem or at the same time) into the vessel 300. In this exemplary dual microcatheter system, the central projection of fluid region (e.g., cone region) of injected glucose solution 125 prevents entry of the injected embolic solution 120 into the outlet port 103b of the inner microcatheter 103. Hence, the inner glucose solution lumen 110 of the inner microcatheter 103 remains unclogged (i.e., free of embolic solution 120) allowing tracking of the guidewire 200 therethrough. Furthermore, the central (inner) projection of fluid (e.g., cone region) of injected glucose solution 125 ensures prevents the injected embolic solution 120 from adhering to the exterior wall (i.e., outer wall 103c) of the inner microcatheter 103. Moreover, the injected embolic solution (e.g., glue) 120 wedged in direct physical contact with the wall of the vessel resists back pressure build-up of injected embolic solution 120 thereby preventing proximal migration. For instance, an adhesive (cured injected embolic solution) wedge may be created by injecting an initial amount of embolic solution locally and waiting until it cures before injecting more embolic solution, so that the first cured portion prevents the second administering of embolic solution from flowing proximally. So, prior to creating the adhesive wedge, glucose solution may be injected to protect the outer wall of the catheter, thereafter, creating the adhesive wedge by injecting an initial portion of embolic solution, waiting for the injected embolic solution to cure and form a wedge, thereafter injecting additional embolic solution. Accordingly, strategic injections of glucose solution may be employed to ensure the embolic solution does not adhere to the catheter.

A modification of the example dual telescopically arranged microcatheters of FIGS. 7A-7C is presented in FIGS. 7D-7F. In FIGS. 7D-7F the distal end/tip 103b of the inner microcatheter 103 is disposed in the longitudinal/axial direction proximally of the distal end/tip of the outer microcatheter 100. The glucose solution 125 is delivered via the outer channel defined between the exterior surface (i.e., outer wall 103c) of the inner microcatheter 103 and the inner wall of the outer microcatheter 100, while the embolic solution 120 is delivered via the embolic solution lumen 105 of the inner microcatheter 103. In this design, the embolic solution 120 is injected or pushed at a higher force through the relatively slower force of injected glucose solution 125 via the outer channel thereby preventing clogging of the distal end/tip 103b of the inner microcatheter 103. During the procedure, the physician may interchange use of the dual microcatheter in FIGS. 7A-7C and the dual microcatheter in FIGS. 7D-7F allowing injection of an initial portion of embolic solution through the central channel distal of outer channel, thereafter advance both channels together through the embolic solution while injecting glucose solution to clear a central lumen through the injected initial embolic solution to prevent that embolic solution from sticking. It would be a relatively small amount of glucose solution to keep the outer diameter mobile without dispersing the injected embolic solution in the first instance. Then the inner channel may be withdrawn in a proximal direction to flow glucose solution directly on the inner channel and ensure it does not get clogged.

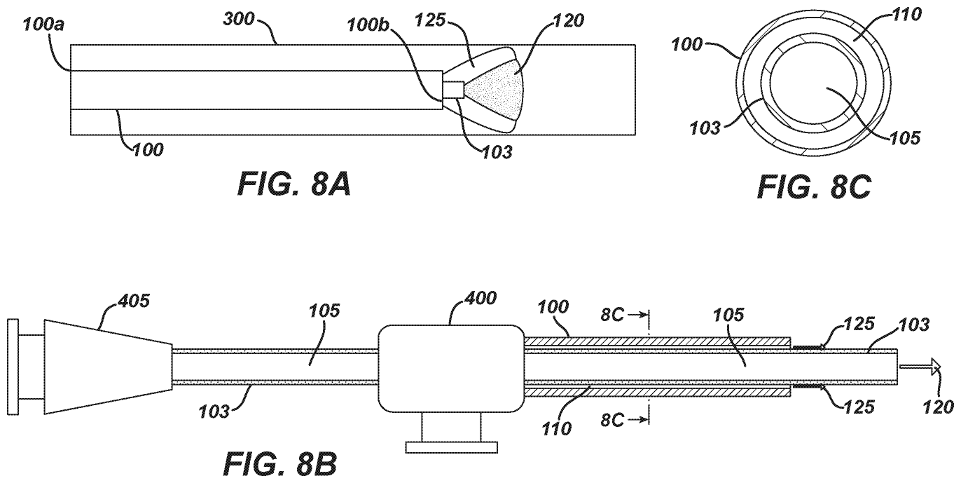

FIG. 8A is a similar configuration of the two telescopically arranged microcatheters of FIG. 7A wherein the distal end/tip 103b of the inner microcatheter 103 is disposed distally relative to the distal end/tip 100b of the outer microcatheter 100, however, the solutions injected in the respective channel/lumen are reversed. That is, in FIG. 8A, the glucose solution 125 delivered via the channel defined between the two microcatheters 100, 103 forms an abluminal projection of fluid (e.g., wedge-shaped funnel) in the vessel 300 radially outward of a central projection of fluid region (e.g., cone) of the embolic solution 120 delivered via the embolic solution lumen 105 of the inner microcatheter 103. The injected embolic solution cures through exposure to ions in blood. So, when the embolic solution is injected through the central channel it projects distally until back pressure pushes it back proximally. The embolic solution is not likely to cure during injection as it is typically a relatively short burst. Rather, the embolic solution cures after or between breaks of injections of embolic solution while the physician is determining if sufficient embolic solution has been dispensed using imagery (e.g., fluoroscopy). If glucose solution is injected from the outer channel continuously or at least after the injection of embolic solution, the injected glucose solution flushes away the blood ions and any dispensed embolic solution from the inner channel. Arrangement in the longitudinal/axial direction of the distal outlet port 100b of the outer catheter 100 distally relative to the distal outlet port 103b of the inner microcatheter 103, causes the proximal injection of the glucose solution 125 to push the injected embolic solution 120 further distally into the vessel beyond that accommodated by the microcatheter and also to prevent adherence of the injected embolic solution 120 to either microcatheter (e.g., exterior surface (outer surface 100c) of the outer microcatheter 100 and/or entering distal outlet port 103b of the inner microcatheter 103).

FIG. 8B is a side view of an exemplary telescopic dispensing system 400 for inducing telescope movement of the respective microcatheters 100, 103 relative to one another. The exemplary telescopic dispensing system 400 includes a telescopic stationary member 405 and a telescopic advanceable member 410. The dual telescopically arranged microcatheters 100, 103 in FIG. 8B are depicted as a longitudinal/axial cross-sectional view. Outer microcatheter 100 is secured in position by the telescopic stationary member 405, while the inner microcatheter 103 secured to the telescopic advanceable member 410 is slidable telescopically (e.g., pushing/pulling) by the physician or interventionalist relative to the stationary outer microcatheter 100.

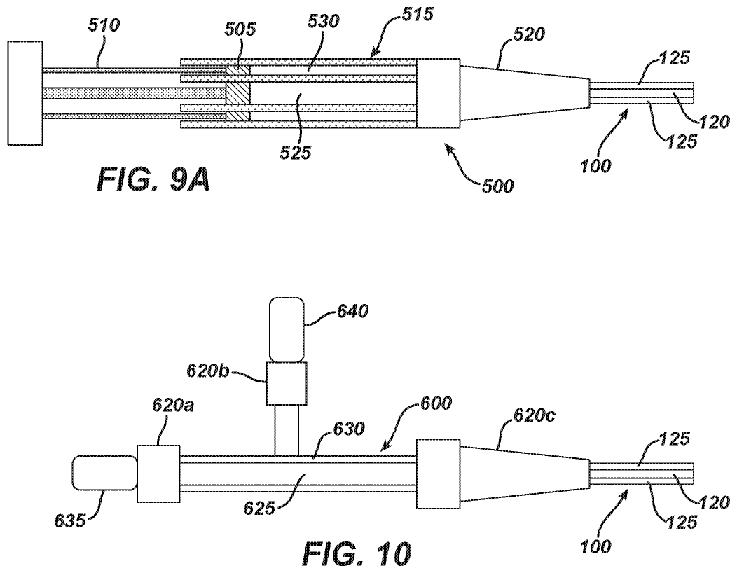

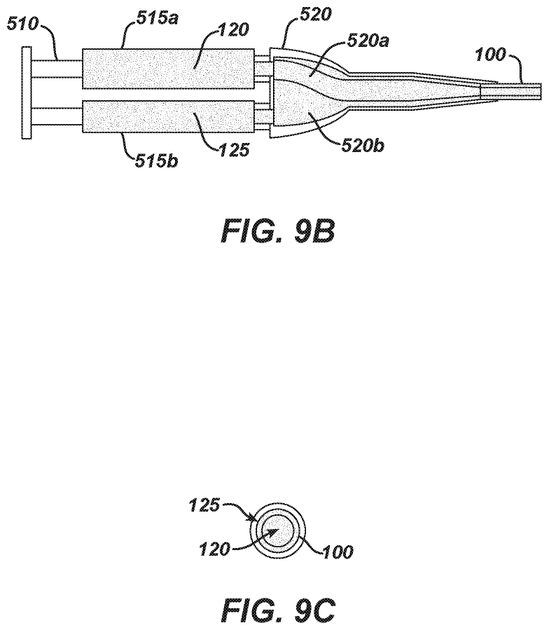

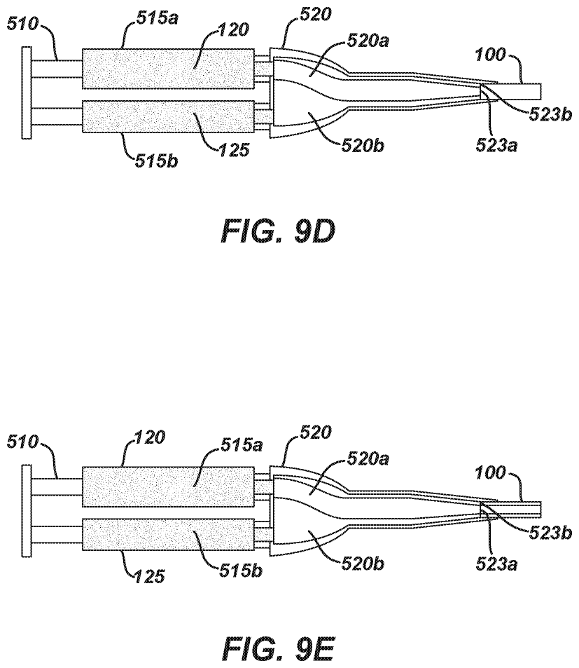

A hub or syringe barrel may be attached to the proximal end of any of the microcatheter configurations illustrated and described herein to deliver the embolic solution 120 and glucose solution 125 either simultaneously (i.e., in tandem or at the same time) or non-simultaneously (i.e., independently, sequentially or not at the same time). Several non-limiting examples of the hub or syringe dispensing system are described in detail. FIG. 9A is a first example dual channel dispensing system 500 including a single barrel syringe 515 having dual concentric channels feeding into a single luer attachment 520 and a slidable multi-walled shaft 510 with a plunger 505 secured to its distal end. When the multi-walled shaft 510 is advanced (i.e., pushed) by the physician or interventionalist the plunger 505 dispenses the solutions 120, 125 in tandem (i.e., simultaneously or at the same time) via the concentrically arranged channels defined in the syringe barrel 515. In particular, the syringe barrel 515 delivers the embolic solution 120 via an inner embolic solution channel 525 and the glucose solution 125 via an outer glucose solution channel 530 disposed radially outward relative to the inner embolic solution channel 525. The microcatheter 100 connected to the distal end of the hub or syringe barrel 500 may have dual lumen (e.g., inner concentric lumen receiving the embolic solution 120 and outer concentric lumen receiving the glucose solution 125). Alternatively, microcatheter 100 may have a single lumen defined therein. In the case of a single lumen microcatheter 100, the embolic and glucose solutions 120, 125, respectively, are preferably injected in tandem with sufficient force (i.e., pressure) to maintain along the entire longitudinal/axial length of the lumen an outer layer of injected glucose solution 125 disposed radially outward of the centrally injected embolic solution 120. This outer layer of injected glucose solution (e.g., dextrose) 125 acts as a buffer layer preventing adherence of the injected embolic solution 120 to the inner liner or inner wall of the lumen. In yet a still further modification (e.g., FIGS. 9B-9E), instead of the single syringe barrel 515 two separate syringe barrels 515a, 515b (arranged side-by-side or parallel to one another) may be employed feeding into a single luer attachment or hub 520 having separate channels 520a, 520b. Referring to FIG. 9B, the first syringe barrel 515a delivering the embolic solution 120 feeds into a first channel 520a of the single hub 520, while the second syringe barrel 515b for delivery of the glucose solution 125 feeds into an outer cavity 520b (i.e., region surrounding the first channel 520a) of the hub 520. The respective channels 520a, 520b of the multi lumen hub 520 (i.e., each lumen separate, independent and distinct from one another) direct the fluids from each barrel 515a, 515b such that glucose solution 125 flows along the inner wall of the single lumen microcatheter 100 while the embolic solution 120 flows centrally through the injected glucose solution (as represented by the distal end view of the single lumen microcatheter 100 in FIG. 9C). In the example of FIG. 9D the respective barrels 515a, 515b are fluidly connected to the respective channels 520a, 520b of the single hub 520. A first of the channels (e.g., channel 520a transporting the embolic solution 120) is disposed centrally (i.e., radially inward) and has a larger distal opening relative to the second channel (e.g., channel 520b transporting the glucose solution 125) radially surrounding (i.e., radially outward) around and having a smaller distal opening relative to the first channel 520a. Distal opening 523b at the distal end of the second channel 520b is aligned with distal opening 523a at the distal end of the first channel 520a. The channels may be switched, i.e., channel 520a transports the glucose solution 125, while channel 520b delivers the embolic solution 120. Microcatheter 100 depicted in FIG. 9D has a single lumen defined therein wherein the embolic and glucose solutions 120, 125, respectively, are preferably injected in tandem with sufficient force (i.e., pressure) maintain along the entire longitudinal/axial length of the lumen an outer layer of injected glucose solution 125 disposed radially outward of the centrally injected embolic solution 120. Alternatively, in FIG. 9E the dual barrel hub (FIG. 9D) may be connected to a concentric dual lumen microcatheter 100 to prevent intermixing between the injected glucose solution 125 and embolic solution 120. The dual lumen microcatheter in FIG. 9E is less flexible than that of the single lumen microcatheter in FIG. 9D.

A simplified example dual lumen hub 600 is shown in FIG. 10 eliminating the multi-wall shaft plunger 510 of the hub 500 FIG. 9A. This dual lumen hub 600 has a first lure connector 620a serving as an inlet port of the inner (central) concentrically arranged embolic solution channel 625 defined longitudinally/axially through the hub for administering the embolic solution 120, and a second (preferably oriented to the side) lure connector 620b in fluid communication with an outer concentrically arranged glucose solution channel 630. Corresponding containers or vessels 635, 640 preloaded with premixed embolic solution and glucose solution, respectively, are fitted to the corresponding lure connector 620a, 620b. Embolic solution 120 and glucose solution 125 is administered via the associated channels 625, 630, respectively, of the dual concentric lumen of the hub 600 either in tandem (i.e., simultaneously) or independently (i.e., non-simultaneously) of one another. Microcatheter 100 connected at its proximal end to the distal end of the dual channel hub 600 in the example of FIG. 10 may have defined therein concentrically arranged channels (e.g., an inner (central) concentric embolic solution channel and an outer concentric glucose solution channel). Alternatively, the dual channel hub 600 in FIG. 10 may be connected to a single lumen microcatheter 100. In the case of a single lumen microcatheter 100, the embolic and glucose solutions 120, 125, respectively, are preferably injected in tandem with sufficient force (i.e., pressure) to maintain along the entire longitudinal/axial length of the lumen an outer layer of injected glucose solution 125 disposed radially outward of the centrally injected embolic solution 120. This outer layer of injected glucose solution (e.g., dextrose) 125 acts as a buffer layer preventing adherence of the injected embolic solution 120 to the inner liner or inner wall of the lumen.

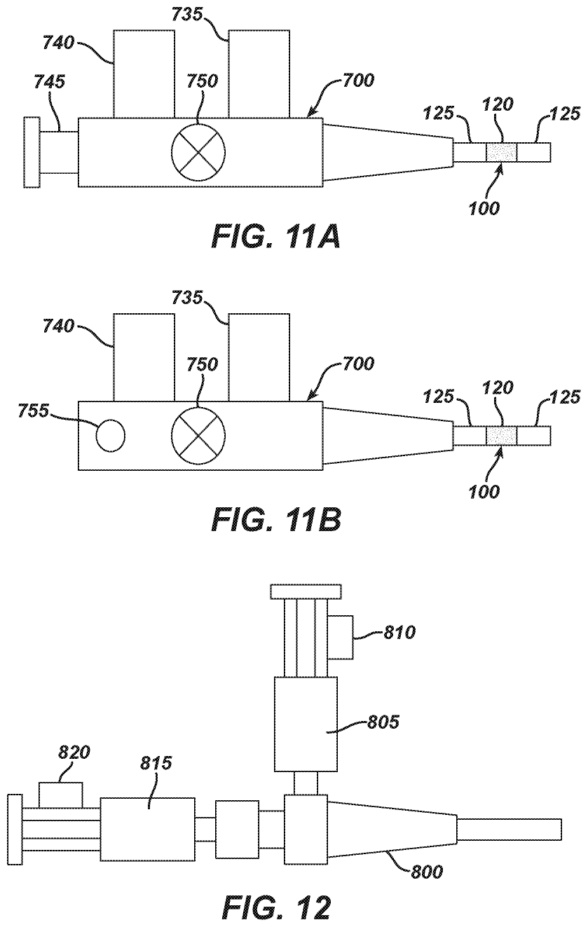

It is also contemplated to use a hub to deliver sequentially the embolic solution 120 and the glucose solution 125 via a single lumen microcatheter. FIG. 11A is a side view of an example hub 700 connected thereto in fluid communication via respective inlet ports (e.g., side inlet ports) are two preloaded containers, vessels or reservoirs (e.g., a glucose solution vessel 735 containing a predetermined volume of premixed glucose solution and an embolic solution vessel 740 containing a predetermined volume of premixed embolic solution 120). In response to the physician or interventionalist manipulating a manual valve 750 (e.g., rotating valve) the sequencing (i.e., timing) of each solution dispensed from an associated vessel into the single lumen microcatheter 100 may be controlled, as desired. For example, the physician or interventionalist may manipulate or control valve 750 to allow flow through the hub 700 of either the embolic solution 120 stored in the embolic solution canister 740 or the glucose solution 125 stored in the glucose solution canister 735. When the valve is an open state for a particular canister to allow flow of the solution stored therein, the volume of that solution dispensed via the hub into the single lumen of the microcatheter 100 is controlled by the physician or interventionalist manually advancing (e.g., pushing) the syringe 445. The volume of solution (e.g., embolic solution or glucose solution) delivered through the single lumen of the microcatheter for example may be a sequence of approximately 0.5 ml of glucose solution 125, approximately 1.0 ml of embolic solution 120, and approximately 0.5 ml of glucose solution 125.

Manual control by the physician or interventionalist of the valve 750 and syringe 745 in the hub 700 of FIG. 11A may alternatively be automated via electronic programming of sequential timing of the opening of the valve 760 to an open state for one of the canisters 735, 740 and dispensing of a desired volume in response to the physician or interventionalist merely actuating a button 755 (FIG. 11B). A processor or controller 765 including an associated memory device stores a desired timing sequence for sequencing and volume control of the solutions from the respective canisters. That is, in response to activating the button 755, the valve 760 is automatically controlled based on the stored timing sequencing automatically controlling via sequencing, timing and duration (i.e., volume) dispensing of the respective embolic solution and the glucose solution delivered through the single lumen of the microcatheter 100.

FIG. 12 is yet another hub 800 in its most basic or simplistic form in which an ampule 805 preloaded with a predefined volume of the embolic solution 120 (e.g., premixture in a desired ratio of an embolic agent and oil) is attached or fitted onto a first lure connector of hub 800. Ampule 805 is sealed thereby preventing premature dispensing of the contents stored therein. For example, ampule 805 may include a corresponding locking tab 810 that when removed, disrupted or broken by the physician or interventionalist dispenses the preloaded premixed solution stored therein. Another sealed ampule 815 preloaded with a predefined volume of premixed glucose solution 125 may be attached in fluid communication to hub 800 via a second lure connector. In response to removing, disrupting or breaking a seal 820 (e.g., locking tab) the contents of ampule 815 may be dispensed. The contents of the embolic solution 120 and glucose solution 125 stored in the ampules 805, 815, respectively, may be dispensed either simultaneously (i.e., in tandem or at the same time) or non-simultaneously (i.e., sequentially, independently or not at the same time). This simplified hub configuration 800 in FIG. 12 provides control only in the timing associated with the removal, disruption or breaking of the seals 810, 815 on the respective ampules 805, 810 and dispensing of solution contained therein. Once the seals 810, 815 are removed, disrupted or broken the entire predefined volume of premixed solution 820, 825, respectively, is dispensed via the hub 800 into the microcatheter 100. Microcatheter 100 connected to the distal end of hub 800 may represent any of the exemplary configurations set forth in the illustrated examples herein and described above, e.g., multi-lumen, dual lumen or single lumen.

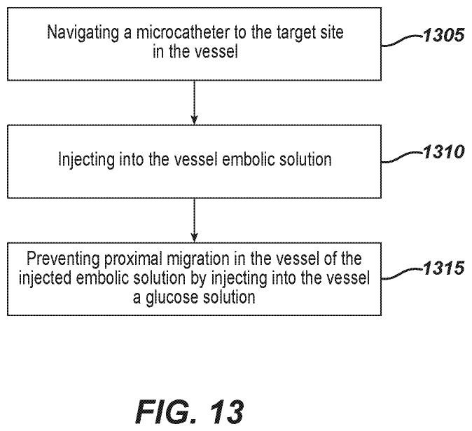

FIG. 13 is an exemplary flow chart of the method of operation of the endovascular embolization system in accordance with the present disclosure wherein glucose solution is injected into the vessel to prevent proximal migration of the injected embolic solution. In step 1305 the microcatheter 100 is navigated to the target site in the vessel. With the microcatheter 100 properly positioned at the target site in the vessel 300, in step 1310 the embolic solution 120 is injected via the microcatheter 100 into the vessel 300 at the target site. To prevent proximal migration in the vessel 300 of the injected embolic solution 120, in step 1315 the glucose solution 125 is injected into the vessel 300.

Aspects of the present disclosure are also provided by the following numbered clauses:
Clause 1: A method for embolization treatment at a target site within a vessel (300) using an endovascular embolization system, the method comprising the steps of: navigating a microcatheter (100) to the target site in the vessel (300); and injecting into the vessel (300) embolic solution (120); and preventing proximal migration in the vessel (300) of the injected embolic solution by injecting into the vessel (300) a glucose solution (125).
Clause 2: The method of Clause 1, wherein the injected glucose solution (125) imparts a force pushing the injected embolic solution distally further into the vessel (300) beyond reach of the microcatheter (100).
Clause 3: The method of any of Clauses 1 through 2, wherein injection of the embolic solution (120) and the glucose solution (125) occurs either simultaneously or non-simultaneously of one another.

Clause 4: The method of any of Clauses 1 through 3, wherein the microcatheter (100) has a proximal end (100a), an opposite distal end (100b) and an outer sidewall (100c) extending longitudinally between the proximal end (100a) and the distal end (100b) defining a first lumen (105) receiving the embolic solution (120) and a second lumen (110) receiving the glucose solution (125), the second lumen (110) being separate from the first lumen (105); wherein the first lumen (105) has a first inlet port (105a) and a first outlet port (105b), while the second lumen (110) has a second inlet port (110a) and a second outlet port (110b); and the injected glucose solution (125) preventing adherence of the embolic solution (120) to a portion of the microcatheter (100).

Clause 5: The method of Clause 4, wherein the second lumen (110) is arranged radially outward relative to the first lumen (105); the injected glucose solution (125) preventing adherence of the injected embolic solution (120) to an exterior surface of the outer sidewall (100c) of the microcatheter (100); and the first lumen (105) and the second lumen (110) are arranged either concentrically or eccentrically relative to one another.

Clause 6: The method of Clause 5, wherein the second outlet port (110 b) of the second lumen (110) is disposed in a longitudinal direction either (i) proximally of the first outlet port (105b) of the first lumen (105); or (ii) aligned with the first outlet port (105b) of the first lumen (105).

Clause 7: The method of Clause 4, wherein the second lumen (110) is arranged radially inward relative to the first lumen (105); the injected glucose solution (125) preventing adherence of the injected embolic solution (120) into the second outlet port (110b) of the second lumen (110); and the first lumen (105) and the second lumen (110) are arranged either concentrically or eccentrically relative to one another.

Clause 8: The method of Clause 7, wherein the second outlet port (110 b) of the second lumen (110) is disposed in a longitudinal direction either: (i) proximally of the first outlet port (105b) of the first lumen (105); or (ii) aligned with the first outlet port (105b) of the first lumen (105).

Clause 9: The method of Clause 4, wherein simultaneously as the injecting step, further comprising aspirating from the vessel (300) at least some of the injected embolic solution (120) and/or the injected glucose solution (125) via an aspiration lumen (115) defined in the microcatheter (100) separate from each of the first lumen (105) and the second lumen (110).

Clause 10: The method of Clause 4, wherein prior to the injecting step, further comprising aspirating blood via an aspiration lumen (115) defined in the microcatheter (100) separate from each of the first lumen (105) and the second lumen (110).

Clause 11: The method of Clause 1, wherein the microcatheter (100) serves as an outer microcatheter having a distal end (100b), an opposite proximal end (100a), an outer sidewall (100c) and a first lumen defined therein in a longitudinal direction; the outer microcatheter (100) is configured to receive in the first lumen an inner microcatheter (103) having a distal end (103b), an opposite proximal end (103a) and an associated second lumen defined therein in the longitudinal direction; a first channel being defined radially between the outer microcatheter (100) and the inner microcatheter (103) and the second lumen representing a second channel.

Clause 12: The method of Clause 11, wherein the first channel receives therein the injected embolic solution (120) and the second channel receives therein the injected glucose solution (125); the distal end (100b) of the outer microcatheter (100) is disposed proximally relative to the distal end (103b) of the inner microcatheter (103); the injected glucose solution (125) preventing adherence of the injected embolic solution (120) into the distal end (103b) of the second lumen of the inner microcatheter (103).

Clause 13: The method of Clause 11, wherein the first channel receives therein the injected glucose solution (125) and the second channel receives therein the injected embolic solution (120); the distal end (103b) of the inner microcatheter (103) is disposed proximally relative to the distal end (100b) of the outer microcatheter (100); the injected glucose solution (125) preventing adherence of the injected embolic solution (120) to an exterior surface of the outer sidewall (100c) of the outer microcatheter (100).

Clause 14: The method of Clause 11; wherein at the same time as the injecting step, further comprising aspirating from the vessel (300) at least some of the injected embolic solution (120) and/or the injected glucose solution (125) via an aspiration lumen (115) defined in the microcatheter (100) separate from each of the first lumen and the second lumen.

Clause 15: The method of Clause 1, wherein the injecting step comprises administering the injected embolic solution (120) and the injected glucose solution (125) via respective separate channels of an administering device (500, 600, 700, 800) fluidly connected to the microcatheter (100).

Clause 16: The method of Clause 15, wherein the administering device (500, 600) is configured to simultaneously deliver into the microcatheter (100) the injected embolic solution (120) disposed radially inward relative to that of the injected glucose solution (125); the injected glucose solution (125) preventing adherence of the injected embolic solution (120) along an inner surface of the microcatheter (100).

Clause 17: The method of Clause 16, wherein the microcatheter (100) has defined therein: (i) a single lumen for receiving both the embolic solution and the glucose solution or (ii) separate lumens for receiving the embolic solution and the glucose solution, respectively.

Clause 18: The method of Clause 15, wherein the microcatheter (100) has a single lumen and the administering device (700) is configured to sequentially deliver into the single lumen of the microcatheter (100) the injected embolic solution (120) and the injected glucose solution (125).

Clause 19: The method of Clause 18, wherein the sequential delivery into the microcatheter (100) of the injected embolic solution (120) and the injected glucose solution (125) occurs via controlling a valve (750) associated with the administering device (700).

Clause 20: The method of Clause 15, wherein the administering device (800) has fluidly connected thereto a first ampule (805) preloaded with a predetermined volume of the embolic solution (120) comprising premixed embolic agent and oil, with premature dispensing of the embolic solution (120) from the first ampule (805) being prevented by an associated seal (810); and the administering device (800) has fluidly connected thereto a second ampule (815) preloaded with a predetermined volume of the glucose solution (125) comprising premixed dextrose and deionized water, with premature dispensing of the glucose solution (125) from the second ampule (815) being prevented by an associated second seal (820); and the injecting step comprises, in response to disrupting the first and second seals (810, 820), dispensing either simultaneously or non-simultaneously the embolic solution (120) from the first ampule (805) and the glucose solution (125) from the second ampule (815) into the microcatheter (100).

Clause 21: An endovascular embolization system comprising: a microcatheter (100) having a first passageway through which an embolic solution (120) is injectable; and separate from the first passageway, the microcatheter (100) further including a second passageway through which a glucose solution (125) is injectable thereby preventing proximal migration of the embolic solution (120) once injected from the microcatheter (100).

Clause 22: The system of Clause 21, wherein the glucose solution (125) once injected from the microcatheter (100) imparts a force pushing further distally the embolic solution (120) once injected from the microcatheter (100).

Clause 23: The system of Clause 21, wherein the embolic solution (120) and the glucose solution (125) are injectable from the microcatheter (100) either simultaneously or non-simultaneously of one another.

Clause 24: The system of Clause 21, wherein the microcatheter (100) has a proximal end (100a), an opposite distal end (100b) and an outer sidewall (100c) extending longitudinally between the proximal end (100a) and the distal end (100b); the first passageway is a first lumen (105) defined in the microcatheter (100) receiving the injectable embolic solution (120) and the second passageway is a second lumen (110) receiving the injectable glucose solution (125), the second lumen (110) being separate from the first lumen (105); wherein the first lumen (105) has a first inlet port (105a) and a first outlet port (105b), while the second lumen (110) has a second inlet port (110a) and a second outlet port (110b); and the injected glucose solution (125) preventing adherence of the embolic solution (120) to a portion of the microcatheter (100).

Clause 25: The system of Clause 24, wherein the second lumen (110) is arranged radially outward relative to the first lumen (105); the injectable glucose solution (125) preventing adherence of the injected embolic solution (120) to an exterior surface of the outer sidewall (100c) of the microcatheter (100); and the first lumen (105) and the second lumen (110) are arranged either concentrically or eccentrically relative to one another.

Clause 26: The system of Clause 25, wherein the second outlet port (110 b) of the second lumen (110) is disposed in a longitudinal direction either (i) proximally of the first outlet port (105b) of the first lumen (105); or (ii) aligned with the first outlet port (105b) of the first lumen (105).

Clause 27: The system of Clause 25, wherein the second lumen (110) is arranged radially inward relative to the first lumen (105); the injected glucose solution (125) preventing adherence of the injected embolic solution (120) into the second outlet port (110b) of the second lumen (110); and the first lumen (105) and the second lumen (110) are arranged either concentrically or eccentrically relative to one another.

Clause 28: The system of Clause 27, wherein the second outlet port (110 b) of the second lumen (110) is disposed in a longitudinal direction either: (i) proximally of the first outlet port (105b) of the first lumen (105); or (ii) aligned with the first outlet port (105b) of the first lumen (105).

Clause 29: The system of Clause 25, wherein separate from each of the first lumen (105) and the second lumen (110), the microcatheter (100) further includes an aspiration lumen (115) through which is received aspirated fluid including at least some of the embolic solution (120) once injected from the microcatheter (100) and/or the glucose solution (125) once injected from the microcatheter (100); wherein the embolic solution (120) being injectable via the first lumen (105), the glucose solution (125) being injectable via the second lumen (110) and the aspirated fluid being aspirated via the aspiration lumen (115) simultaneously of one another.

Clause 30: The system of Clause 25, wherein separate from each of the first lumen (105) and the second lumen (110), the microcatheter (100) further includes an aspiration lumen (115) through which is received aspirated fluid including at least some of the embolic solution (120) once injected from the microcatheter (100) and/or the glucose solution (125) once injected from the microcatheter (100); wherein the aspirated fluid being aspirated via the aspiration lumen (115) prior to the embolic solution (120) being injectable via the first lumen (105) together with the glucose solution (125) being injectable via the second lumen (110).

Clause 31: The system of Clause 21, wherein the microcatheter (100) serves as an outer microcatheter having a distal end (100b), an opposite proximal end (100a), an outer sidewall (100c) and a first lumen defined therein in a longitudinal direction; the outer microcatheter (100) is configured to receive in the first lumen an inner microcatheter (103) having a distal end (103b), an opposite proximal end (103a) and an associated second lumen defined therein in the longitudinal direction; the first passageway is a first channel being defined radially between the outer microcatheter (100) and the inner microcatheter (103) and the second lumen representing the second passageway is a second channel.

Clause 32: The system of Clause 31, wherein the first channel receives therein the injectable embolic solution (120) and the second channel receives therein the injectable glucose solution (125); the distal end (100b) of the outer microcatheter (100) is disposed proximally relative to the distal end (103b) of the inner microcatheter (103); the injectable glucose solution (125) preventing adherence of the injectable embolic solution (120) into the distal end (103b) of the second lumen of the inner microcatheter (103).

Clause 33: The system of Clause 31, wherein the first channel receives therein the injectable glucose solution (125) and the second channel receives therein the injectable embolic solution (120); the distal end (103b) of the inner microcatheter (103) is disposed proximally relative to the distal end (100b) of the outer microcatheter (100); the injectable glucose solution (125) preventing adherence of the injected embolic solution (120) to an exterior surface of the outer sidewall (100c) of the outer microcatheter (100).

Clause 34: The system of Clause 31, wherein separate from the first lumen and the second lumen, the microcatheter (100) further includes an aspiration lumen (115) receiving therein aspirated fluid including at least some of the embolic solution (120) once injected from the microcatheter (100) and/or the glucose solution (125) once injected from the microcatheter (100).

Clause 35: The system of Clause 21, further comprising an administering device (500, 600, 700, 800) having respective separate channels fluidly connected to administer into the microcatheter (100) the injectable embolic solution (120) and the injectable glucose solution (125).

Clause 36: The system of Clause 35, wherein the administering device (500, 600) is configured to simultaneously deliver into the microcatheter (100) the injected embolic solution (120) disposed radially inward relative to that of the injected glucose solution (125); the injected glucose solution (125) preventing adherence of the injected embolic solution (120) along an inner surface of the microcatheter (100).

Clause 37: The system of Clause 36, wherein the microcatheter (100) has defined therein: (i) a single lumen for receiving both the injectable embolic solution (120) and the injectable glucose solution (125) or (ii) separate lumens for receiving the injectable embolic solution (120) and the injectable glucose solution (125), respectively.

Clause 38: The system of Clause 35, wherein the microcatheter (100) has a single lumen and the administering device (700) is configured to sequentially deliver into the single lumen of the microcatheter (100) the injectable embolic solution (120) and the injectable glucose solution (125).

Clause 39: The system of Clause 38, wherein the administering device (700) further comprises a valve (750) controllable for the sequential delivery into the microcatheter (100) of the injectable embolic solution (120) and the injectable glucose solution (125).

Clause 40: The system of Clause 35, wherein the administering device (800) has fluidly connected thereto a first ampule (805) preloaded with a predetermined volume of the embolic solution (120) comprising premixed embolic agent and oil, with premature dispensing of the embolic solution (120) from the first ampule (805) being prevented by an associated seal (810); and the administering device (800) has fluidly connected thereto a second ampule (815) preloaded with a predetermined volume of the glucose solution (125) comprising premixed dextrose and deionized water, with premature dispensing of the glucose solution (125) from the second ampule (815) being prevented by an associated second seal (820); the first and second seals (810, 820) being disruptable, dispensing either simultaneously or non-simultaneously the embolic solution (120) from the first ampule (805) and the glucose solution (125) from the second ampule (815) into the microcatheter (100).

The descriptions contained herein are examples of embodiments of the invention and are not intended in any way to limit the scope of the invention. As described herein, the invention contemplates many variations and modifications of a method for embolization treatment at a target site within a vessel using an endovascular embolization system wherein proximal migration in the vessel of injected embolic solution is prevented by injecting glucose solution into the vessel. Modifications and variations apparent to those having skilled in the pertinent art according to the teachings of this disclosure are intended to be within the scope of the claims which follow.

What is claimed is:

1. A method for embolization treatment at a target site within a vessel using an endovascular embolization system, the method comprising the steps of:
navigating a microcatheter to the target site in the vessel; and
injecting into the vessel embolic solution; and
preventing proximal migration in the vessel of the injected embolic solution by injecting into the vessel a glucose solution.

2. The method of claim 1, wherein the injected glucose solution imparts a force pushing the injected embolic solution distally further into the vessel beyond reach of the microcatheter.

3. The method of claim 2, wherein injection of the embolic solution and the glucose solution occurs either simultaneously or non-simultaneously of one another.

4. The method of claim 1, wherein the microcatheter has a proximal end, an opposite distal end and an outer sidewall extending longitudinally between the proximal end and the distal end defining a first lumen receiving the embolic solution and a second lumen receiving the glucose solution, the second lumen being separate from the first lumen; wherein the first lumen has a first inlet port and a first outlet port, while the second lumen has a second inlet port and a second outlet port; and the injected glucose solution preventing adherence of the embolic solution to a portion of the microcatheter.

5. The method of claim 4, wherein the second lumen is arranged radially outward relative to the first lumen; the injected glucose solution preventing adherence of the injected embolic solution to an exterior surface of the outer sidewall of the microcatheter; and the first lumen and the second lumen are arranged either concentrically or eccentrically relative to one another.

6. The method of claim 5, wherein the second outlet port of the second lumen is disposed in a longitudinal direction either (i) proximally of the first outlet port of the first lumen; or (ii) aligned with the first outlet port of the first lumen.

7. The method of claim 4, wherein the second lumen is arranged radially inward relative to the first lumen; the injected glucose solution preventing adherence of the injected embolic solution into the second outlet port of the second lumen; and the first lumen and the second lumen are arranged either concentrically or eccentrically relative to one another.

8. The method of claim 7, wherein the second outlet port of the second lumen is disposed in a longitudinal direction either: (i) proximally of the first outlet port of the first lumen; or (ii) aligned with the first outlet port of the first lumen.

9. The method of claim 4, wherein simultaneously as the injecting step, further comprising aspirating from the vessel at least some of the injected embolic solution and/or the injected glucose solution via an aspiration lumen defined in the microcatheter separate from each of the first lumen and the second lumen.

10. The method of claim 4, wherein prior to the injecting step, further comprising aspirating blood via an aspiration lumen defined in the microcatheter separate from each of the first lumen and the second lumen.

11. An endovascular embolization system comprising: a microcatheter having a first passageway through which an embolic solution is injectable; and separate from the first passageway, the microcatheter further including a second passageway through which a glucose solution is injectable thereby preventing proximal migration of the embolic solution once injected from the microcatheter.

12. The system of claim 11, wherein the glucose solution once injected from the microcatheter imparts a force pushing further distally the embolic solution once injected from the microcatheter.

13. The system of claim 11, wherein the embolic solution and the glucose solution are injectable from the microcatheter either simultaneously or non-simultaneously of one another.

14. The system of claim 11, wherein the microcatheter has a proximal end, an opposite distal end and an outer sidewall extending longitudinally between the proximal end and the distal end; the first passageway is a first lumen defined in the microcatheter receiving the injectable embolic solution and the second passageway is a second lumen receiving the injectable glucose solution, the second lumen being separate from the first lumen; wherein the first lumen has a first inlet port and a first outlet port, while the second lumen has a second inlet port and a second outlet port; and the injected glucose solution preventing adherence of the embolic solution to a portion of the microcatheter.

15. The system of claim 14, wherein the second lumen is arranged radially outward relative to the first lumen; the injectable glucose solution preventing adherence of the injected embolic solution to an exterior surface of the outer sidewall of the microcatheter; and the first lumen and the second lumen are arranged either concentrically or eccentrically relative to one another.

16. The system of claim 15, wherein the second outlet port of the second lumen is disposed in a longitudinal direction either (i) proximally of the first outlet port of the first lumen; or (ii) aligned with the first outlet port of the first lumen.

17. The system of claim 15, wherein the second lumen is arranged radially inward relative to the first lumen; the injected glucose solution preventing adherence of the injected embolic solution into the second outlet port of the second lumen; and the first lumen and the second lumen are arranged either concentrically or eccentrically relative to one another.

18. The system of claim 17, wherein the second outlet port of the second lumen is disposed in a longitudinal direction either: (i) proximally of the first outlet port of the first lumen; or (ii) aligned with the first outlet port of the first lumen.

19. The system of claim 15, wherein separate from each of the first lumen and the second lumen, the microcatheter further includes an aspiration lumen through which is received aspirated fluid including at least some of the embolic solution once injected from the microcatheter and/or the glucose solution once injected from the microcatheter; wherein the embolic solution being injectable via the first lumen, the glucose solution being injectable via the second lumen and the aspirated fluid being aspirated via the aspiration lumen simultaneously of one another.

20. The system of claim 15, wherein separate from each of the first lumen and the second lumen, the microcatheter further includes an aspiration lumen through which is received aspirated fluid including at least some of the embolic solution once injected from the microcatheter and/or the glucose solution once injected from the microcatheter ; wherein the aspirated fluid being aspirated via the aspiration lumen prior to the embolic solution being injectable via the first lumen together with the glucose solution being injectable via the second lumen.
